(19) United States
(12) Patent Application Publication
(10) Pub. No.: US 2007/0155012 A1
(43) Pub. Date: Jul. 5, 2007

(54) Method for production of neurons from cells of a cell line

(75) Inventors: Alain Privat, St. Clement de Riviere (FR); Sophie Marchal, Villeneuve les Magulone (FR); Jean-Philippe Hugnot, Montpellier (FR)

Correspondence Address: EGBERT LAW OFFICES, 412 MAIN STREET, 7TH FLOOR, HOUSTON, TX, 77002, US

(73) Assignee: Alain Privat, St. Clement de Riviere (FR)

(21) Appl. No.: 10/570,098

(22) PCT Filed: Sep. 1, 2004
(86) PCT No.: PCT/FR04/50406

§ 371 Date: Oct. 21, 2006

(30) Foreign Application Priority Data
Sep. 2, 2003 (FR) 0310382

(51) Int. Cl. C12N 15/09; C12N 5/08; C12N 5/06

(52) U.S. Cl. 435/455; 435/368; 435/325

(57) ABSTRACT

The invention relates to a method for production of neurons from cells of a cell line which may be differentiated to produce neurons in particular, whereby said cells are cultivated in spheres, preferably by exposing the same to growth factors, such as, for example, EGF (epidermal growth factor) and/or bFGF (basic fibroblast growth factor) or LIF (Leukemia Inhibitory Factor), in a given growth medium, the differentiation in said spheres is induced on forcing the same to adhere to a substrate, after removal of the growth factors EGF and/or bFGF or LIF, and cultivating the same in the growth medium for an appropriate duration. Said method is characterised in that cells of the human embryonic teratocarcinoma NT2 are used.

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### FIELD OF THE INVENTION

This invention relates to a method for producing neurons from cells of a human cell line capable of differentiating in order to produce namely neurons, in which:
said cells are cultivated into spheres, by exposing them to growth factors, such as, for example, EGF (epidermal growth factor) and/or bFGF (basic fibroblast growth factor) or LIF (Leukemia Inhibitory Factor), in a specified growth medium,
differentiation of said spheres is induced by causing them to adhere on a substrate, after elimination of growth factors EGF and/or bFGF or LIF, and by cultivating them in the growth medium for an appropriate period of time.

The invention also relates to the use, for various applications, of neurons stemming from the implementation of this method.

### BACKGROUND OF THE INVENTION

Numerous research laboratories now work on elaborating techniques aimed at permitting both the comprehension and the mastery of the functions of the central and peripheral nervous system, mostly for therapeutic purposes, but also simply for the purpose of obtaining useful research evolution models.

Thus, the development of the methods for producing neurons falls within the scope, in particular, of the projects for elaborating cell therapies that, with the transplant of pluripotent and/or progenitive stem cells, constitute a promising alternative permitting to consider the replacement of any cells of the spinal cord and of the brain that might have been destroyed and re-create an environment favorable to nerve regeneration.

Mastering neuron production is therefore a hope of healing for numerous patients suffering from lesions of the spinal cord, or from neurodegenerative diseases, the most obvious consequences of which are characterized by dysfunctions in the transmission of nerve signals sent by the brain to the peripheral structures, that can lead, in extreme cases, to paralyses together with sensory deficits.

Furthermore, the fact that one can have human neurons produced in laboratory in great quantity can also considerably favor the carrying out of studies conducted in vitro on molecules of therapeutic importance, and permits one to consider an advantageous model within the scope of research on genes that are important for the development of the central and peripheral nervous system.

One of the techniques currently used in order to produce neurons is based on the property of pluripotentiality of the stem neural cells, which, as described by Gage and al. (Current Opinion in Neurobiology 1998, 8:671-676), are brought, after a cultivation phase in the presence of growth factors leading to spherical aggregates, to differentiate into neurons and glia after adhesion on a medium and elimination of growth factors.

Although the neural stem cells are regarded as advantageous because of their lack of carcinogenic risks, and though they constitute nowadays the object of a great number of researches, this technique still offers only narrow prospects, because the differentiation of these cells after transplantation leads almost exclusively to the production of glial cells, i.e. of astrocytes and of oligodendrocytes. to the detriment of the production of neurons which constitute only 1 to 5% of all cells obtained.

Such a small yield obviously does not permit one to consider a re-implantation of neurons in a possible lesion.

In addition, it is known that astrocytes, after a transplant, are likely to limit the growth of the neurons and to secrete molecule es modifying negatively the environment of transplanted cells.

Other known methods also consist in obtaining neurons after differentiation of cells of the human embryonic teratocarcinoma line (NT2) treating them with retinoic acid.

One of them, described in particular by Andrews and at. (Developmental Biology 1984, 103:285-293), consisting in cultivating the NT2 cellular line into monolayer, thus leads to the production of 5% mature post-mitotic neurons, of which a succession of re-cultures in specific conditions permits eventual ly a 99% purification of neurons NT2-N.

This neuron producing technique is however tong, tedious and has the disadvantage of a considerable loss of material during the various re-cultures.

In addition, treatment with retinoic acid presupposes the use of bovine serum, involving, for some applications, a potential risk of spongiform bovine encephalitis, or of hepatitis.

Another known method described by Cheung and Al (BioTechniques 1999, 26:946-954), based on the preliminary formation of cell aggregates from NT2 cells, although having the advantage of reducing time required for the technique of cultivation into monolayers to bring about neuronal differentiation, also presupposes the use of bovine serum, and therefore, the possibility of risks as described above.

Looking for solutions capable of coping with the various disadvantages, the inventors of this method have found that cells of the human embryonic teratocarcinoma line, treated on the basis of the method used for the differentiation of the neural stem cells, but in very specific and scrupulously elaborated conditions, were able, quite unexpectedly and astonishingly, to produce a particularly considerable percentage of neurons, without loss of material, and in complete safety, since the contemplated solution does not presuppose the use of bovine serum anymore.

### BRIEF SUMMARY OF THE INVENTION

Accordingly, this invention now constitutes a practical solution for the various applications exposed above, and therefore permits one to seriously consider their development.

In fact, the invention generally relates to a method for producing neurons from cells of a cell line capable of differentiating in order to produce in particular neurons, in which:
said cells are cultivated into spheres, preferably by exposing them to growth factors, such as, for example, EOF (epidermal growth factor) and/or bFGF (basic fibroblast growth factor) or LIF (Leukemia Inhibitory Factor), in a specified growth medium, and
differentiation of said spheres is induced by causing them to adhere on a substrate, after elimination of growth factors EGF and/or bFGF or LIFs, and by cultivating them in the growth medium for an appropriate period of time, characterized in that cells of the human embryonic teratocarcinoma NT2 line are used.

According to a preferred embodiment, this method essentially comprises three phases: a first step of induction, a step of expansion, both in volume and in number, of spheres derived from the induction, and finally a step of differentiation into neurons.

For the step of induction of cells of the human embryonic teratocarcinoma line (NTI) into spheres:
cells of the human embryonic teratocarcinoma line (NT2), cultivated into monolayers are dissociated with a trypsin/EDT A solution,
NT2 cells are cultured, once dissociated, preferably at 100 000 cells/ml in flasks, for example of the FALCON™ type, of 75 ml, with a filtering plug containing the growth medium to which is added extemporaneously growth factor EGF and/or growth factor bFGF, and
they are left to proliferate for a period of at least seven days.

According to another advantageous feature of the method considered, a specified growth medium is used, not containing bovine serum.

In order to carry out the step of expansion, a fraction of the growth medium is renewed on a regular basis during the period of culturing NT2 cells into spheres.

This is preferably achieved through renewing every three to four days 70% of the growth medium.

Another feature of said method is furthermore defined by the fact that during the period of culturing NT2 cells into spheres, neurospheres in suspension in the growth medium are subjected to centrifugation on a regular basis, and they are taken out by mechanical dissociation, carried out, for example, by means of a tapered Pasteur pipette.

It was found that, quite advantageously, the current conditions permitted to culture NT2 spheres more than 6 times over a period of 60 days, without loss of material.

In order to induce the differentiation of NT2 spheres, this method uses poly-D-lysine (POL), preferably of small molecular weight (for example 30 kDa to 70 kDa) as substrate capable of causing NT2 cell spheres to adhere and differentiate.

On the other hand, according to an embodiment in order to carry out the differentiation of NT2 cell spheres, the latter are cultured on the adhesive substrate without previously dissociating them.

In this case, the method contemplates culturing non-dissociated NT2 cell spheres at 50000-100000 cells/cm² estimating their number by counting an aliquot.

According to another embodiment, in order to carry out the differentiation of NT2 cell spheres, NT2 cell spheres are first dissociated into single cells before culturing them on the adhesive substrate.

Preferably, the method then contemplates to carry out the dissociation of NT2 cell spheres by incubating them for several minutes in a trypsin/EDTA solution, then by exposing them to a solution containing 2 mM CaCl₂ 0.01% DNase 1 and 0.5% trypsin inhibitor.

An additional feature also consists in culturing NT2 cell spheres once dissociated at 250000 cells/cm² on the adhesive substrate, estimating their number by counting an aliquot.

Furthermore, this method is also characterized in that during the phase of differentiation of NT2 spheres, the latter are cultivated for at least ten days.

According to another advantageous feature, this method also contemplates, prior to the differentiation phase, freezing entire NT2 cell spheres (without any preliminary dissociation) in a freezing environment, defined by the growth medium NS in which they have grown (conditioned medium) enriched with the presence of 10% Dimethyl Sulfoxide (DMSO), then defrosting them in a defrosting environment defined by a mixture comprising preferably 50 vol. % of the conditioned medium and 50 vol. % of the new growth medium NS, in the presence of growth factors bFGF and/or EGF or LIF.

Other objectives and advantages of this invention will appear in the course of the following description referring to an example of embodiment, given indicatively and not restrictively.

### BRIEF DESCRIPTION OF THE SEVERAL VIEWS OF THE DRAWINGS

The comprehension of this description will be facilitated by the attached drawings.

FIGS. 1 and 2 are photographs corresponding to phase contrast pictures illustrating the evolution of NT2 cells through the course of this method.

FIG. 3 is a graph illustration representing results from studies regarding the response of NT2 cells to growth factors FGF, bFGF, and LIF.

FIG. 4 is another photograph representing a phase contrast picture of differentiated NT2 spheres.

FIGS. 5 and 6 are photographs representing results from immunofluorescence analyses performed on differentiated NT2 spheres.

FIG. 7 is a photograph representing a western blot showing, on differentiated NT2 spheres, the expression of a neuron-specific marker, 1-13 tubulin.

### DETAILED DESCRIPTION OF THE INVENTION

The invention relates to the field of neurology and provides a new method for obtaining neurons, in which cells of the human embryonic teratocarcinoma line NT2 are cultivated into spherical aggregates then induced to differentiate into neurons after adhesion on a substrate.

In a preliminary step of this method, cells of the cell line NT2 are first cultivated in a classical way, into monolayers, in flasks having filtering plugs containing an Opti-MEM™ (trademark registered by the Life Technologies company) growth medium completed with 5% fetal calf serum, and 5 μg/ml Gentamicin™ (trademark registered by the Gibeo BRL company) at 37° C.

In order to maintain the line, cells cultivated into mono layers are dissociated twice a week into single cells with a 0.25% trypsin/EDTA solution and taken out at one third.

For the implementation of this method, NT2 cells cultivated into monolayers are retrieved and dissociated with the 0.25% trypsin/EDTA solution.

This step permits to obtain the first passing over into spheres of NT2 cells that are then cultured, preferably at 100000 cells/ml in 75 ml flasks of the FALCON™ (trademark registered by the Falcon company) type having filtering plugs containing 15 ml of growth medium (NS) not containing bovine serum, defined by the following composition: DMEM/F12 (50%/50%), 2 mM glutamine, N2 complement, 0.6% glucose, 20 μg/ml insulin, to which are added extemporaneously growth factors EGF at 20 ng/ml, bFGF at 10 ng/ml, 2 μg/ml heparin or growth factor LIF at 10 ng/ml or 20 n/ml.

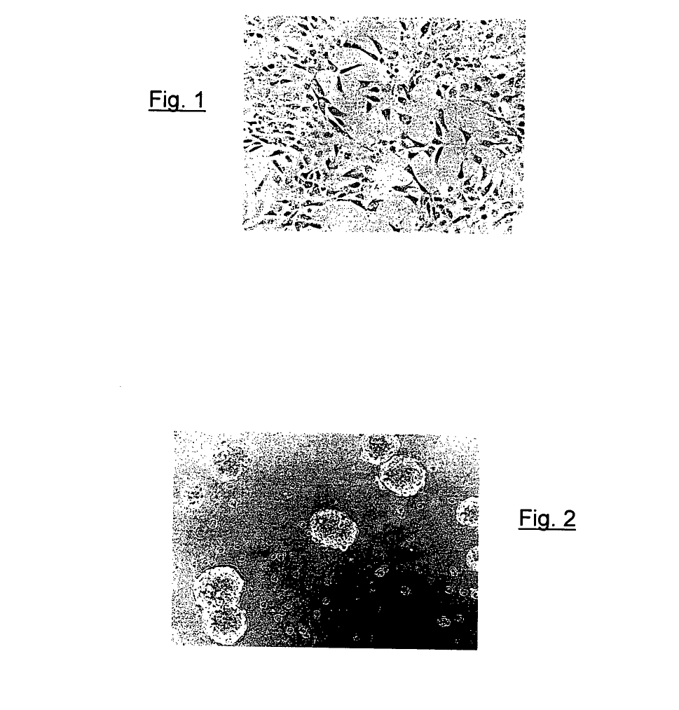

In these cultures, shown in FIG. 1, cells dissociated into single cells do not adhere strongly to the plastic support of the culture flasks.

After 2 days, cells form small spherical aggregates detaching themselves from the plastic and floating in suspension in the growth medium NS, as shown in FIG. 2.

These spheres continue to increase in size and number for 7 to 10 days, and, in order to maintain the line, those among them that are present in suspension in the growth medium NS are centrifuged once a week and taken out by mechanical dissociation carried out by means of a tapered Pasteur pipette, at the rate of 10 to 12 cycles.

Under such circumstances, cells are re-cultured more than 6 times over a period of 60 days, through adding growth factors, or through renewing the growth medium NS, preferably at the rate of 70%, every three to four days.

The way NT2 cells respond to growth factors EGP and bFGF was studied by counting viable spherical cells obtained after three days of culture in the presence either of the one or the other, either of the one and the other of them then dissociated.

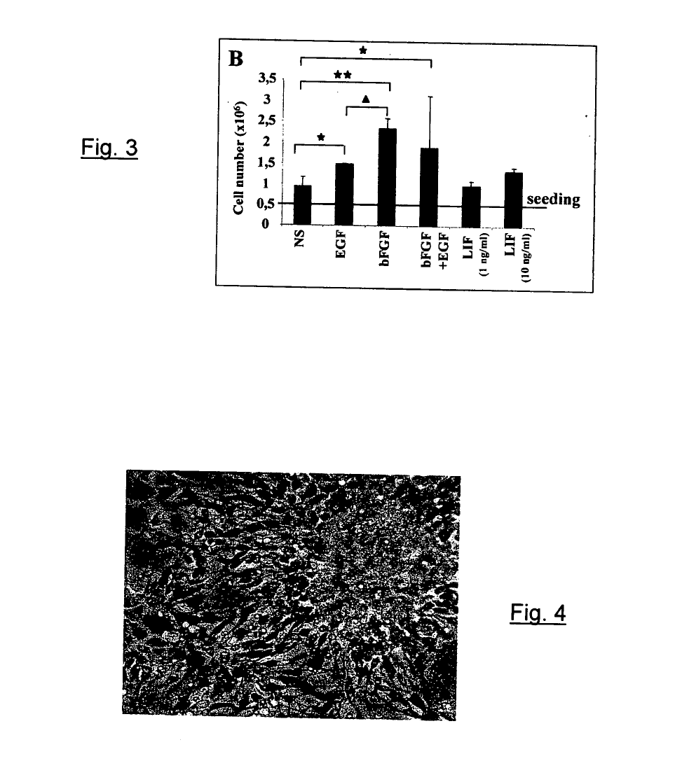

Results represented in FIG. 3 show the number of viable cells after three days, whereas the horizontal line indicates culture density.

Although it was found, unexpectedly, that NT2 cells were capable of forming spheres in the absence of growth factors, they proliferate differently under the action of the latter, and according to the type of growth factors added to the growth medium NS.

Thus, it was found that in the presence of, exclusively, growth factor EGF, the rate of proliferation of NT2 cells is multiplied by 1.5 with respect to results corresponding to cultures without growth factor.

This rate is multiplied by 2.2 in the presence of growth factor bFGF exclusively, whereas the joint presence of the two factors does not show any additional action.

According to this method, in order to carry out the differentiation of NT2 spheres, the media from the culture flasks containing NT2 spheres are centrifuged after 7 to 10 days of proliferation, then residues are washed twice with Phosphate buffer (phosphate-buffered saline, PBS) in order to eliminate any trace of growth factors EGF and bFGF or LIF.

Non-dissociated cells arc then distributed at 50000-100000 cells/cm² either on 24-well plates containing glass strips covered with poly-D-lysine (PDL) at 40 μ/ml, or on culture boxes of 15 mm in diameter covered with PDL at 40 μl/ml.

They are cultivated in these conditions for 10 days without changing the medium.

According to another alternative designed to permit accurate evaluation of the percentage of differentiated cells, NT2 spheres are subjected to dissociation into single cells, by means of a (0.25%) trypsin/EDTA solution in the presence of 2 mM CaCl_{2,} 0.01% DNase 1 and 0.5% trypsin inhibitor, before being cultured on 24-well plates containing glass strips covered with PDL.

Upon treating NT2 spheres in this way, it was observed that they differentiated spontaneously after the withdrawal of growth factors, and adhesion on PDL.

The latter is performed in less than 24 hours and is accompanied by the appearance of two cell types leaving the spheres.

After 10 days of differentiation, the different visible morphologies in FIG. 4 appear, on the one hand cells that are flat and very large, and on the other hand smaller neuronal cells, bipolar and more compact. Studies have also been carried out in order to verify the characteristics of pluripotentiality of NT2 spheres, and the presence of neuronal cells after differentiation.

Thus, the expression of the markers specific to neurons (β3 tubulin, Map2ab), oligodendrocytes (O4) and astrocytes (GFAP) was studied by means of immunofluorescence, performed on differentiated NT2 spheres.

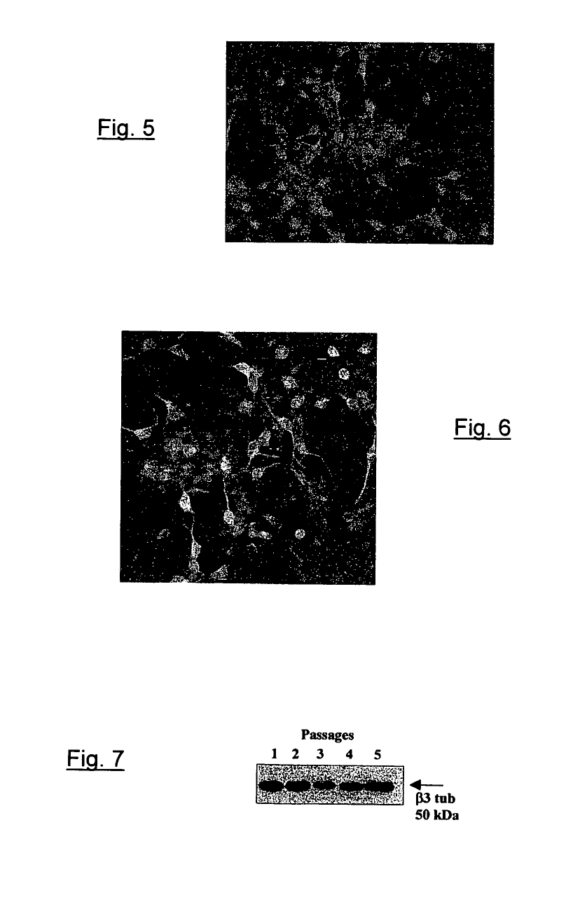

Results obtained from this analysis conducted in a classical way show that after 10 days of differentiation, 30 to 50% of cells are β3 tubulin (FIG. 5), or Map2ab positive (FIG. 6), and that at this stage, no cell expresses O4 and GFAP.

The expression of certain neurotransmitters has also been studied by means of immunofluorescence, and it showed that GABA was the major neurotransmitter, followed by dopamine and serotonin.

All these results show therefore that the method according to the invention leads to the exclusive production of neurons, contrary to the technique based on the use of neural stem cells, and that in addition, this production provides a great quantity of neurons, contrary to the method consisting in treating NT2 cells with retinoic acid.

Furthermore, since NT2 spheres have the advantage of being able to be dissociated for more than two months. It was also established that in the course of successive passages, they preserved the same capability of differentiating into neurons.

For this reason, total proteins of differentiated NT2 spheres were extracted for each passage, and the expression of β3 tubulin was studied at these stages by means of western blot.

The results, visible in FIG. 7, show that the expression of this neuronal marker is still very strong, from the first to the fifth passage, which corresponds to a period extending over more than two months, and therefore that NT2 spheres do not show any loss of their potentiality of neuronal differentiation during the dissociations.

In addition, NT2 cell spheres can be frozen intact (without any preliminary dissociation) in the growth medium NS in which they have grown (conditioned freezing medium) in the presence of 10% Dimethyl Sulfoxide (DMSO).

This advantageously permits to make a stock of NT2 spheres with low passage.

Finally, NT2 cell spheres frozen intact are defrosted in a conditioned medium and new growth medium NS (50%/50%) in the presence of growth factors bFGF and/or EGF or LIF.

In this case, spheres break up a little for 3 to 4 days, then start to proliferate normally, and can again be dissociated into single cells from 7 to 10 days after the day of defrosting.

As it was clearly demonstrated above, the method according to the invention has numerous advantages with respect to methods used classically in order to produce neurons.

NT2 cells cultivated into spheres permit to produce a considerable amount of neurons, and constitute a particularly advantageous model for studying the early development of the human central nervous system and the neurogenesis, directly from human tissue, contrary to the usual practice primarily based on the use of rat and mouse neurons, in particular because of unavailability of primary human neurons.

Thus, neurons obtained can be advantageously used for selecting new agents, in particular protein molecules and/or factors that are supposed to intervene in the differentiation of neural stem cells, and acting so as to favor proliferation of neurons, to the detriment of other types of neuronal cells. Having at disposition such agents is essential, in particular in view of transplantation.

The same neurons can al so be used for selecting agents, acting at the level of the growth of neurites, and that could be used within the scope of repairing strategies, in order to favor re-growth of damaged neurons.

Another interesting application of neurons obtained through this method, relates to their use for sifting agents that can have neuroprotective properties, i.e. capable of protecting neurons from aggressions of different nature, such as, for example, those stemming from certain free radicals, or those as a result of an excitotoxicity phenomenon, of the glutamatergic type or other type. Further, neurons obtained can be used for evaluating intrinsic neurotoxicity of molecules for therapeutic purposes that can get in contact with the central nervous system. They permit therefore the selection of potentially therapeutic agents not having intrinsic toxicity for neurons of the central nervous system.

On the other hand, due to the absence of bovine serum throughout the method, NT2 cells also constitute an extremely promising solution for producing neurons that can be used for obtaining grafts permitting to contemplate in complete safety a transplantation in many pathologies, in particular neurodegenerative diseases, cerebral vascular accidents, traumas of the spinal cord and of the brain, pathologies of the retina or of the inner ear.

Finally, another important advantage is defined by the fact that NT2 cells cultivated in this way do not generate astrocytes, which eliminates any problems connected with limitation of the growth of neurons and secretion of molecules modifying negatively the environment of transplanted cells.

Although the invention has been described with reference to a particular embodiment, it is obviously not limited thereto at all, and various modifications can be made as to forms, materials and combinations of these different elements without departing from the scope and from the spirit of the invention.

We claim:

1. Method for producing neurons from cells of a cell line capable of differentiating in order to produce in particular neurons, said method comprising the steps of:
cultivating said cells into spheres by exposure of said cells to growth factors, such as, EGF (epidermal growth factor) and/or bFGF (basic fibroblast growth factor) or LIF (Leukemia Inhibitory Factor), in a specified growth medium; and
differentiating said spheres induced by making said spheres adhere on a substrate, after elimination of growth factors EGF and/or bFGF or LIF, and by cultivating said spheres in the growth medium for a period of time, wherein cells of a human embryonic teratocarcinoma line NT2 are used.

2. Method according to claim 1, wherein said step of cultivating said cells of the human embryonic teratocarcinoma line (NT2 ) into spheres comprises:
cultivating said cells of the human embryonic teratocarcinoma line (NT2 ) into monolayers dissociated with a (0.25%) trypsin/EDTA solution;
culturing NT2 cells, once dissociated, being preferably at 100 000 cells/ml in culture flasks containing the growth medium, to which is added extemporaneously growth factor EGF and/or growth factor bFGF or LIP; and
proliferating the cells for a period of at least seven days.

3. Method according to claim 1, further comprising;
using a specified growth medium, not containing bovine serum.

4. Method according to claim 1, further comprising;
renewing, during said step of culturing NT2 cells into spheres, a fraction of the growth medium on a regular basis.

5. Method according to claim 4, wherein said step of renewing is 70% of the growth medium is renewed every three to four days 70% of the growth medium.

6. Method according to claim 1, further comprising:
subjecting said NT2 spheres, during the period of culturing NT2 cells into spheres, in suspension in the growth medium, to centrifugation on a regular basis, and
taking out said NT2 spheres by mechanical dissociation, performed by a tapered Pasteur pipette.

7. Method according to claim 1, wherein said step of differentiating NT2 spheres, is comprised of:
using poly-D-lysine (PDL) of small molecular weight 30kDa to 70 kDa, as substrate capable of causing NT2 cell spheres to adhere and differentiate.

8. Method according to claim 1, wherein said step of differentiating NT2 spheres, is comprised of;
culturing said spheres on adhesive substrate without dissociating said spheres beforehand.

9. Method according to claim 8, further comprising:
culturing non-dissociated NT2 cell spheres at 50000-100000 cells/cm estimating a number thereof by counting an aliquot.

10. Method according to claim 1, wherein said step of differentiating NT2 cell spheres, is comprised of:
dissociating first the NT2 cell spheres into single cells before culturing said cells on said adhesive substrate.

11. Method according to claim 10, wherein said step of dissociating the NT2 spheres is comprised of:
incubating the spheres for several minutes in a (0.25%) trypsin/EDTA solution; and
exposing said spheres to a solution containing 2 mM CaCl_{2,} 0.01% DNase 1 and 0.5% trypsin inhibitor.

12. Method according to claim 10, wherein said step of dissociating the NT2 spheres is comprised of:
spreading at 250000 cells/cm² on the adhesive substrate, estimating a number thereof by counting an aliquot.

13. Method according to claim 1, wherein said step of differentiating NT2 spheres, is comprised of:
cultivating the spheres for at least ten days.

14. Method according to claim 1, further comprising:
freezing, prior to said step of differentiating, entire NT2 cell spheres and without any preliminary dissociation, in a freezing environment, defined by the growth medium NS in which the spheres have grown in a conditioned medium, enriched with presence of 10% Dimethyl Sulfoxide (DMSO); and
defrosting in a defrosting environment defined by a mixture comprising preferably 50 vol. % of a conditioned medium and 50 vol. % of new growth medium NS, in presence of growth factors bFGF and/or EGF or LIF.

15. A method of using neurons stemming from the implementation of the method according to claim 1, for obtaining grafts implanted within a scope of treatment of certain pathologies, in particular neurodegenerative diseases, cerebral vascular accidents, traumas of the spinal cord and of the brain, diseases of the retina or of the inner ear.

16. A method of using neurons stemming from implementation of said method according to claim 1, for selecting agents, such as protein molecules and/or factors that can intervene in the differentiation of neural stem cells.

17. A method of using neurons stemming from implementation of the method according to claim 1, for selecting agents, such as protein molecules or factors that can participate in the process of growth of neurites.

18. A method of using neurons stemming from implementation of the method according to claim 1, for selecting agents that can have neuroprotective properties.

19. A method of using neurons stemming from implementation of the method according to claim 1, for selecting potentially therapeutic agents not having toxicity for neurons of the central nervous system.
